(19) United States
(12) Patent Application Publication
(10) Pub. No.: US 2025/0213238 A1
(43) Pub. Date: Jul. 3, 2025

(54) SURGICAL RETRACTOR INSTRUMENT SECUREMENT SYSTEM

(71) Applicant: JGMG BENGOCHEA, LLC, Jacksonville, FL (US)

(72) Inventors: Javier GARCIA-BENGOCHEA, Jacksonville, FL (US); John Souza, SR., Monroe, NC (US)

(73) Assignee: JGMG BENGOCHEA, LLC, Jacksonville, FL (US)

(21) Appl. No.: 19/005,487

(22) Filed: Dec. 30, 2024

Related U.S. Application Data
(60) Provisional application No. 63/616,447, filed on Dec. 29, 2023.

(51) Int. Cl. A61B 17/02

(52) U.S. Cl. CPC A61B 17/0206; A61B 17/0218

(57) ABSTRACT

A surgical instrument securement system for use with a surgical retractor includes a bracket comprising first and second opposing bracket arms, and an instrument securement base that is releasably engageable with one or both of the first and second opposing bracket arms, the instrument securement base comprising an instrument retention seat, the instrument retention seat configured to retain a portion of at least one surgical instrument. The bracket and instrument securement base assembly are configured to secure the at least one surgical instrument to a surgical retractor adjacent at least a portion of the interior surface of the surgical retractor.

### RELATED APPLICATIONS

This application claims the benefit of and priority to U.S. Prov. App. No. 63/616,447, entitled “SURGICAL RETRACTOR INSTRUMENT SECUREMENT SYSTEM,” filed Dec. 29, 2023, which is hereby incorporated by reference in its entirety.

### FIELD

The present application describes various exemplary devices, systems and methods for facilitating visualization and protecting nerves and other soft tissue while accessing a surgical site within the body, particularly the spine. More particularly, the present application describes devices, systems and methods useful for securing soft tissue away from the surgical field within a surgical retractor placed adjacent spinal tissue, for example a tubular retractor.

### DESCRIPTION OF THE RELATED ART

A problem with known retractors used in minimally invasive surgery, particularly tubular retractors, is that they present a small portal to the surgical site, and due to uneven contact with soft and bony tissue the small portal is prone to blockage due to tissue creep, and in particular, creep of critical tissues such as nerves which must be gently retracted and capable of being released to avoid trauma and damage to the nerve tissue. The small size of the portal limits the space for introducing conventional blade and other types of manipulators that could be used to move tissue from the site.

### SUMMARY

The shortcomings in the art have been overcome by the instant invention which provides a system and individual devices and methods that enable fixed retraction of soft tissue out of the field of view through the surgical retractor.

In various embodiments, the invention includes a surgical instrument securement system for use with or integral with a surgical retractor, which may be in some embodiments a cylindrical or tubular retractor, and an instrument securement base and a bracket. In various embodiments the surgical instrument securement system includes a bracket that extends from and may be affixed to or integral with a surgical retractor, and an instrument securement base. In some embodiments the instrument securement base includes a clamp that includes an integral instrument retention seat. In some embodiments the instrument securement base includes stacked arcuate supports and a slidable locking collar that includes an integral instrument retention seat.

In some embodiments, the invention also includes at least one extension clip that is affixable to a surgical retractor, for example a tubular retractor, the retractor having a body that is adapted for securing the extension clip within and adjacent an inner wall to extend from the end of the surgical retractor into contact with soft tissue for contacting and displacing soft tissue away from the surgical field. The extension clip may be affixed via snap fitting within slots in the retractor or engagement via an upper hook and a lower hook that is affixed on respective proximal and distal ends of the retractor. The extension clip is suitable for use with tubular as well as curved blade and planar retractor systems, though as shown in the drawings herein, the surgical retractor is a tubular retractor of the sort used for minimally invasive spinal surgery.

As described herein, in some particular embodiments, the invention includes an instrument securement base that includes a clamp with bracket and the affixable instrument shown herein as a nerve root retractor, although the clamp with bracket could also be used to secure other instruments to the surgical retractor such as suction, irrigation, lighting, and other instruments for contacting or manipulating tissue. In some embodiments, the inventive system is referred to as an assembly, and in some embodiments the inventive system when together with the surgical retractor is referred to as an assembly. And in some embodiments, at least the bracket of the inventive system is integral with the surgical retractor.

In various embodiments the disclosure provides a surgical instrument securement system for use with a surgical retractor, comprising a bracket comprising first and second opposing bracket arms; and an instrument securement base that is releasably engageable with one or both of the first and second opposing bracket arms, the instrument securement base comprising an instrument retention seat, the instrument retention seat configured to retain a portion of at least one surgical instrument. The bracket and instrument securement base, when assembled, are configured to secure the at least one surgical instrument to a surgical retractor that has an elongate body having a proximal end, a distal end, an interior surface, and an exterior surface, and a retractor axis defined along the elongate body from the proximal to the distal end. When the at least one surgical instrument is releasably secured to the surgical retractor, the at least one surgical instrument is positioned adjacent at least a portion of the interior surface of the surgical retractor.

In some embodiments, the surgical retractor is generally cylindrical in shape.

In some embodiments, the surgical retractor includes affixed clip extensions for contacting and displacing soft tissue away from the surgical field.

In some embodiments, at least one of the first and second opposing bracket arms includes an emplacement for retaining one or more of surgical instruments selected from nerve retractors, tissue shims, lighting attachments, suction attachments, or a combination thereof.

In some embodiments, the bracket is integral with the surgical retractor.

In some embodiments, the bracket is configured to releasably attach to a handle of the surgical retractor.

In some embodiments, the bracket comprises a central plate positioned between the opposing bracket arms, the plate including on a back side a pair of opposing recesses for releasable receiving the handle of the surgical retractor.

In some embodiments, the bracket comprises an elastically deformable plate having a front side and a back side, and first and second rails extending from the back side of the elastically deformable plate, each of the first and second rails including at least one receptacle, the first rail including at least a first receptacle and the second rail including at least a second receptacle, the first receptacle on the first rail disposed facing the second receptacle on the second rail, wherein the first and second receptacles are arranged to receive and secure the handle of the surgical retractor.

In some embodiments, the first and second opposing bracket arms adjacent to and extending from the front side of the elastically deformable plate are flexible such that the first and second opposing bracket arms are actuatable to flex towards each other to deform the elastically deformable plate and expand a distance between the first and second rails to thereby receive and secure the handle of the surgical retractor in the first and second receptacles or remove the retractor arm from the first and second receptacles.

In some particular embodiments, the instrument securement base comprises a clamp comprising first and second compression wings, each of the first and second opposing compression wings comprising a releasable lock, each of the first and second compression wings configured to receive and releasably compress, respectively, the first and second opposing bracket arms, and the instrument retention seat.

In some embodiments, the releasable lock on each of the first and second compression wings of the clamp includes opposing and interfitting flange and tab extensions.

In some embodiments, each of the first and second compression wings comprises opposing compression branches comprising an upper compression branch and a lower compression branch, and wherein the releasable lock on each of the first and second compression wings of the clamp includes opposing and interfitting flange and tab extensions positioned at a distal end of each of the first and second compression wings.

In some embodiments, each of the upper compression branches comprises a tab extension and each of the lower compression branches comprises a flange extension, and wherein when engaged, the tab extensions of each of the upper branches engage with the flange extensions of each of the lower branches to compress and secure the clamp to the opposing bracket arms.

In some particular embodiments, the instrument securement base comprises a clamp in which each of the first and second compression wings comprises opposing compression branches comprising an upper compression branch and a lower compression branch, and wherein the releasable lock on each of the first and second compression wings includes opposing and interfitting flange and tab extensions positioned central to the clamp adjacent the instrument retention seat which is positioned between the first and second compression wings.

In some embodiments, the flange of releasable lock on each of the first and second compression wings extends from the clamp and is oriented perpendicular to the compression wings, and tab extension of the releasable lock on each of the first and second compression wings of the clamp is located at a central terminus of each of the upper compression branches.

In some embodiments, the instrument retention seat comprises a single recess for receiving a portion of a surgical instrument.

In some embodiments, the instrument retention seat comprises at least one recess for receiving a generally cylindrical portion of a surgical instrument handle.

In some particular embodiments, the instrument securement base comprises stacked arcuate supports comprising upper and lower arcuate arms, each upper and lower arcuate arms comprising at least one securement bar for engagement with one of the emplacement of the bracket arms; and a slidable locking collar that is engageable with an arcuate arm of the stacked arcuate supports, the slidable locking collar comprising the instrument retention seat for securing at least one surgical instrument to the stacked arcuate supports, wherein, the slidable locking collar is slidably engageable along the upper arcuate arm to enable selectable positioning of an engaged surgical instrument for positioning along a selected portion of the interior surface of the surgical retractor

In another embodiment, the disclosure provides a kit for securing a surgical instrument to surgical retractor, comprising: a surgical instrument securement system according to claim 1;
at least one surgical instrument selected from nerve retractors, tissue shims, lighting attachments, suction attachments, or a combination thereof; and a cylindrical surgical retractor comprising a handle.

In another embodiment, the disclosure provides a method for accessing the surgical field in a patient, comprising: providing surgical instrument securement system; affixing the surgical instrument securement system to a tubular surgical retractor in a surgical field to provide a surgical retractor assembly; passing a selected surgical instrument into the tubular surgical retractor, and as determined by the surgeon, the maneuvering a distal end of the surgical instrument for into contact with tissue below/adjacent a distal end of the surgical retractor so as to initially displace soft tissue away from the distal end of the surgical retractor; securing the instrument securement by engagement with the instrument retention seat of the instrument securement base; locking the instrument to the instrument retention seat; optionally, intermittently releasing the instrument from the instrument securement base; and upon completion of the surgical procedure, removing the surgical retractor assembly.

### BRIEF DESCRIPTION OF THE DRAWINGS

Features and advantages of the general inventive concepts will become apparent from the following description made with reference to the accompanying drawings, including drawings represented herein in the attached set of figures, of which the following is a brief description:

FIG. 1 shows an embodiment of the clamp with bracket system as affixed to a tubular surgical retractor that is held by a table arm 80 fixator, the clamp with bracket shown engaged with and fixing an elongate instrument, as shown a nerve root retractor, within and against the wall of the tubular retractor as it would be situated in the surgical filed adjacent a spinal disc space, the inventive system and tubular retractor together referred to herein as a first assembly including the clamp with bracket;

FIG. 2 shows a side view of the first assembly including the clamp with bracket in which the clamp is closed securing it to the bracket arms;

FIG. 3 shows a top view of the first assembly including the clamp with bracket;

FIG. 4 shows a side perspective view of the first assembly including the clamp with bracket in which the clamp is in an open configuration not yet locked and secured to the bracket arms;

FIG. 5 shows a closeup front view of the first assembly including the clamp with bracket in which the clamp is in an open configuration not yet locked and secured to the bracket arms;

FIG. 6 shows another top view of the first assembly including the clamp with bracket in which the clamp is closed securing it to the bracket arms;

FIG. 7 shows another perspective view of the first assembly including the clamp with bracket in which the clamp is closed securing it to the bracket arms;

FIG. 8 shows another front view of the first assembly including the clamp with bracket in which the clamp is closed securing it to the bracket arms;

FIG. 9 shows another top view of the first assembly including the clamp with bracket in;

FIG. 10 shows top perspective, side, and top views the clamp without the bracket in which the clamp is in an open configuration not yet locked;

FIG. 11 shows top perspective, side, and top views the clamp without the bracket in which the clamp is in a locked configuration;

FIG. 12 shows a top perspective view of another embodiment of the instrument securement base according to the instant invention in a second assembly, wherein the instrument securement base is configured to lock in an alternate manner as compared to the first instrument securement base embodiment;

FIG. 13 shows a front view of the assembly in FIG. 12;

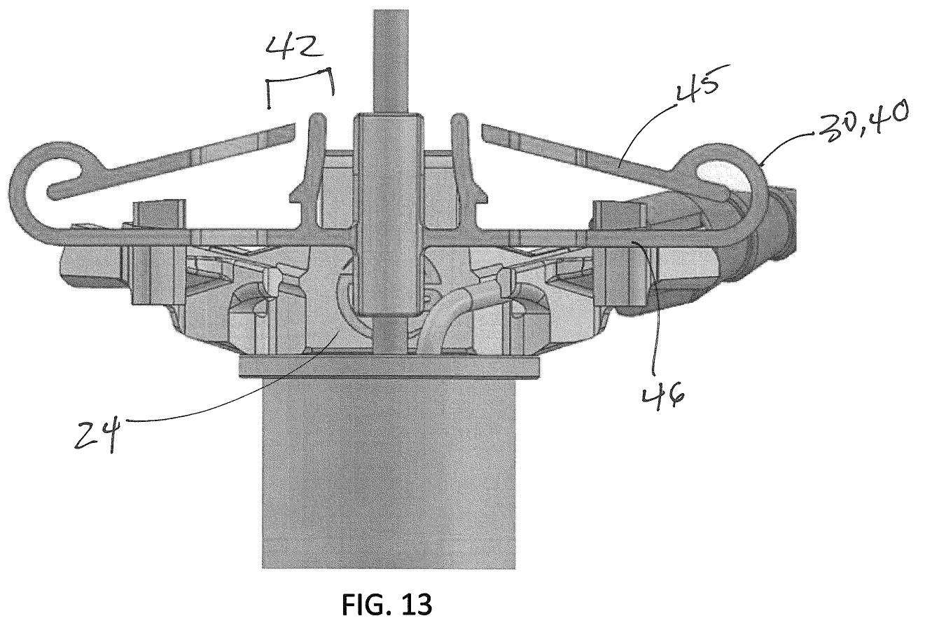

FIG. 14 shows side and tip views of the clamp from the assembly in FIG. 12;

FIG. 15 shows an alternate embodiment of a bracket with arms that is affixable directly to the table arm (shown with the bracket from FIGS. 1-9 and 12-13 in the background);

FIG. 16 is an alternate top perspective view of the assembly shown in FIG. 12;

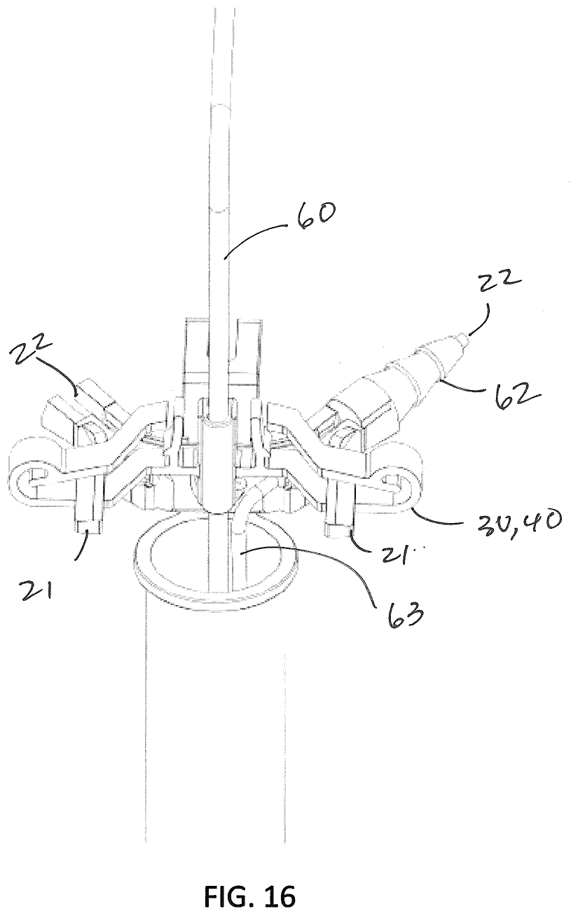

FIG. 17 shows a top perspective view of another embodiment according to the instant invention in a second assembly, wherein the instrument securement base includes a pair of stacked arcuate supports to which the nerve root retractor instrument may be variably coupled as shown with a locking collar gripping the upper arc and securing the elongate instrument handle in a securement channel of the locking collar;

FIG. 18 is a top perspective view of the instrument securement base from FIG. 17;

FIG. 19 is a side view of the instrument securement base from FIG. 17;

FIG. 20 is a top view of the instrument securement base from FIG. 17;

FIG. 21 is a side perspective view of the instrument securement base from FIG. 17; and

FIG. 22 includes examples of clip extensions.

### PARTS LIST

| | |
|---|---|
| surgical instrument securement system | 10 |
| bracket | 20 |
| first & second opposing bracket arms | 21 |
| emplacement | 22 |
| central plate | 23 |
| front side | 24 |
| back side | 25 |
| opposing recesses | 26 |
| first and second rails | 27 |
| first receptacle | 28 |
| second receptacle | 29 |
| instrument securement base | 30 |
| clamp | 40 |
| first and second compression wings | 41 |
| compression wing distal end | 49 |
| releasable lock | 42 |
| tab extension | 43 |
| flange extension | 44 |
| upper compression branch | 45 |
| lower compression branch | 46 |
| central terminus of each of the upper | 47 |
| compression branches | |
| stacked arcuate supports | 50 |
| upper and lower arcuate arms | 51 |
| securement bar | 52 |
| slidable locking collar | 53 |
| instrument retention seat | 31 |
| surgical instrument | 60 |
| surgical instrument handle | 61 |
| suction tubing adapters | 62 |
| suction tubing | 63 |
| nerve root retractor | 64 |
| clip extensions | 65 |
| surgical retractor | 70 |
| handle | 71 |
| elongate body | 72 |
| proximal end | 73 |
| distal end | 74 |
| interior surface | 75 |
| exterior surface | 76 |
| retractor axis | 77 |
| table arm | 80 |

### DETAILED DESCRIPTION

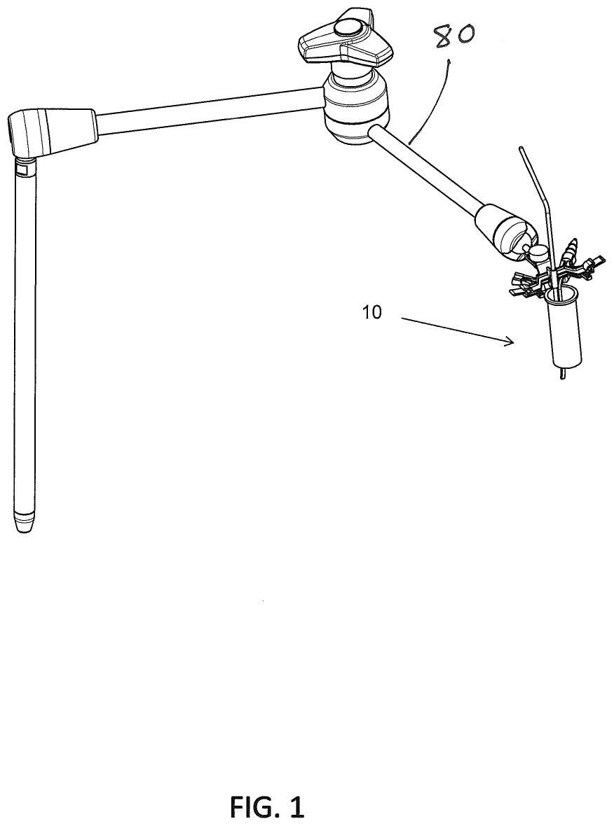

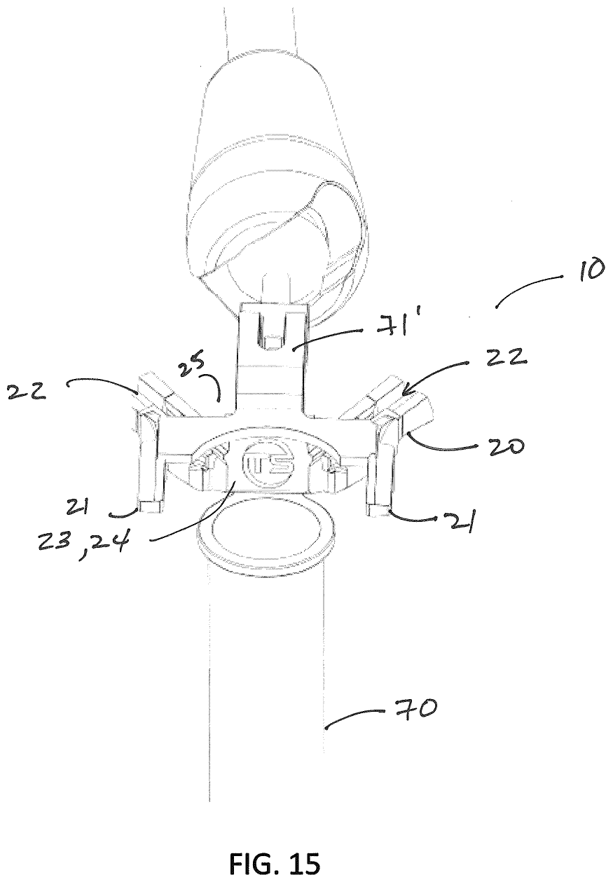

In various embodiments, the disclosure provides a surgical instrument securement system 10 for use with a surgical retractor 70, in some embodiments a tubular retractor. Referring to FIG. 1, the surgical instrument securement system 10 is suitable for use in a surgery, for example a spinal surgery, wherein the retractor 70 is affixed to a stabilized table arm 80 for maintaining the position of the retractor 70 in the surgical field. The surgical instrument securement system 10 includes a bracket 20 comprising first and second opposing bracket arms 21, as shown in FIG. 15, where the bracket 20 is shown affixed to the surgical retractor 70 and affixed directly to the table arm 80. The surgical instrument securement system 10 also includes an instrument securement base 30 that is releasably engageable with one or both of the first and second opposing bracket arms 21, the instrument securement base 30 comprising an instrument retention seat 31, the instrument retention seat 31 configured to retain a portion of at least one surgical instrument 60. The bracket 20 and instrument securement base 30, when assembled, are configured to secure the at least one surgical instrument 60 to a surgical retractor 70. Referencing for example, FIG. 15, the surgical retractor 70 may be a cylindrical tube type retractor, or may include one or a plurality of blades. The surgical retractor 70 has an clongate body 72 having a proximal end 73, a retractor distal end 74, an interior surface 75, and an exterior surface 76, and a retractor axis 77 defined along the elongate body 72 from the retractor proximal end 73 to the retractor distal end 74. When the at least one surgical instrument 60 is releasably secured to the surgical retractor 70, the at least one surgical instrument 60 is positioned adjacent at least a portion of the interior surface 75 of the surgical retractor 70.

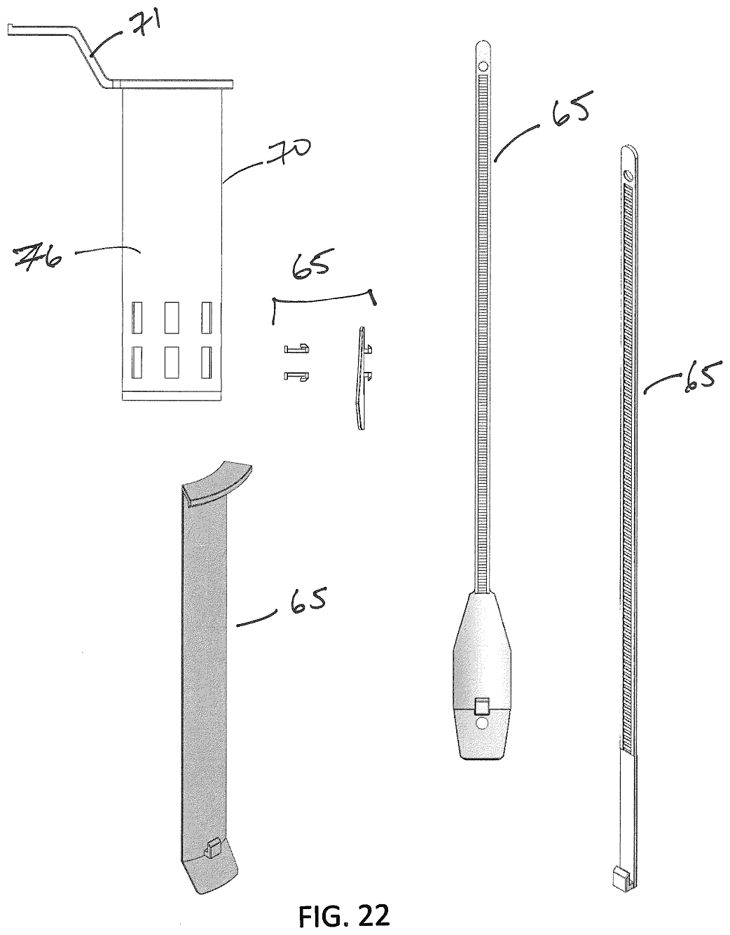

Referring now to FIG. 22, in some embodiments, the surgical instrument securement system 10 includes clip extensions 65 that may be affixed to the surgical retractor 70 for contacting and displacing soft tissue away from the surgical field. As shown, in some embodiments the surgical retractor 70 is adapted with features that accept corresponding features on a clip that may be inserted therein for retention on the surgical retractor 70, wherein the attachments may be by means of slots and corresponding tabs, or one or more hooks that may extend from the distal end 74 of the surgical retractor 70 and may be secured by engagement with the instrument securement base 30.

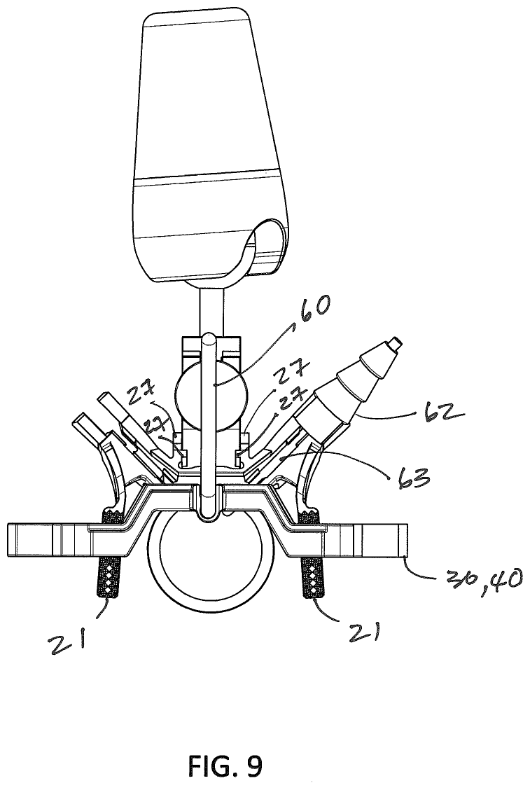

Referring now to FIG. 9, for example, in some embodiments of the surgical instrument securement system 10, at least one of the first and second opposing bracket arms 21 includes an emplacement 22 for retaining one or more of surgical instruments 60 selected from nerve retractors, tissue shims, lighting attachments, suction attachments, or a combination thereof.

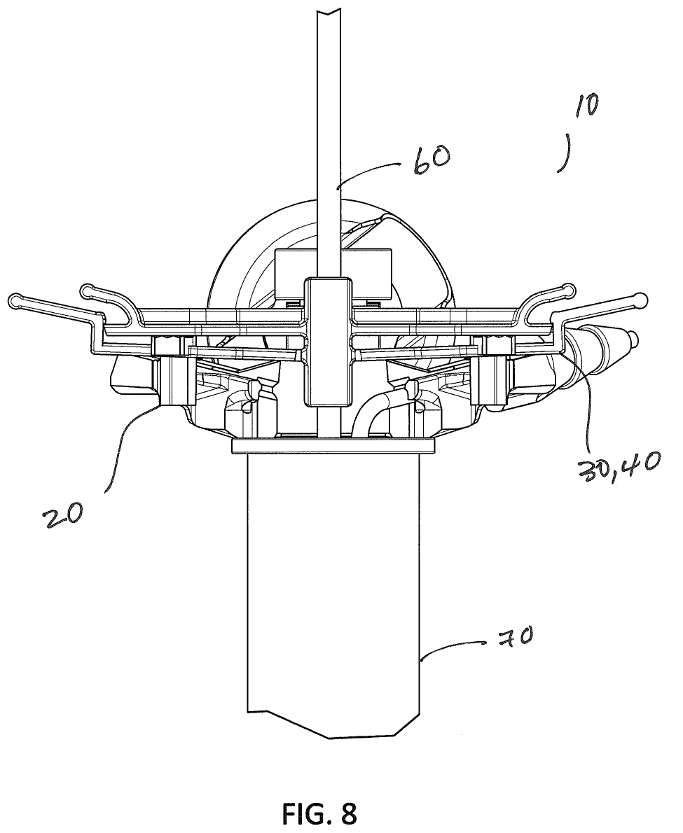

As shown in the drawings, for example in FIG. 9, the emplacements 22 on the bracket may be adapted to retain one or more surgical instruments or fixtures, such as suction tubes or fiber lighting, and may thus include suction tubing adapters 62 as show, for example, in FIG. 9 and emplaced suction tubing 63 as shown, for example, in FIG. 8.

In some embodiments of the surgical instrument securement system 10, the bracket 20 is integral with the surgical retractor 70 (not shown). In some embodiments, as shown variously in the drawings, the bracket 20 is configured to releasably attach to a handle 71 of the surgical retractor 70.

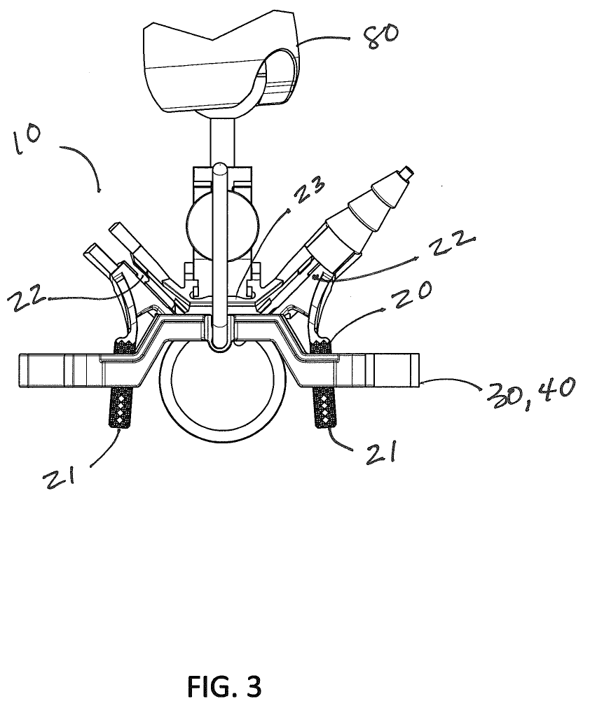

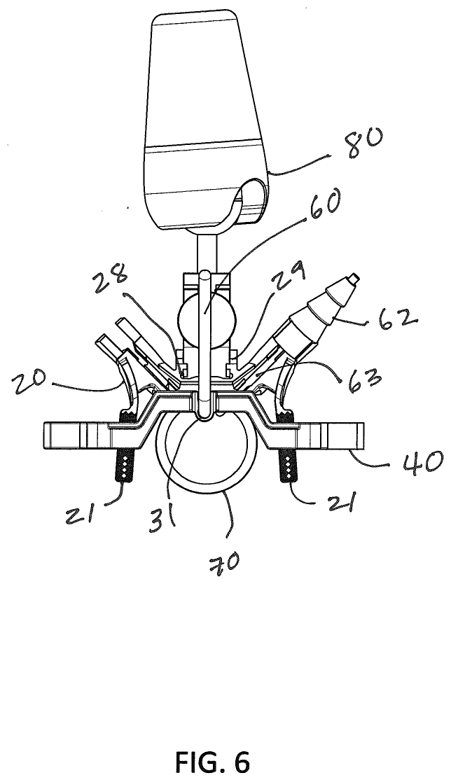

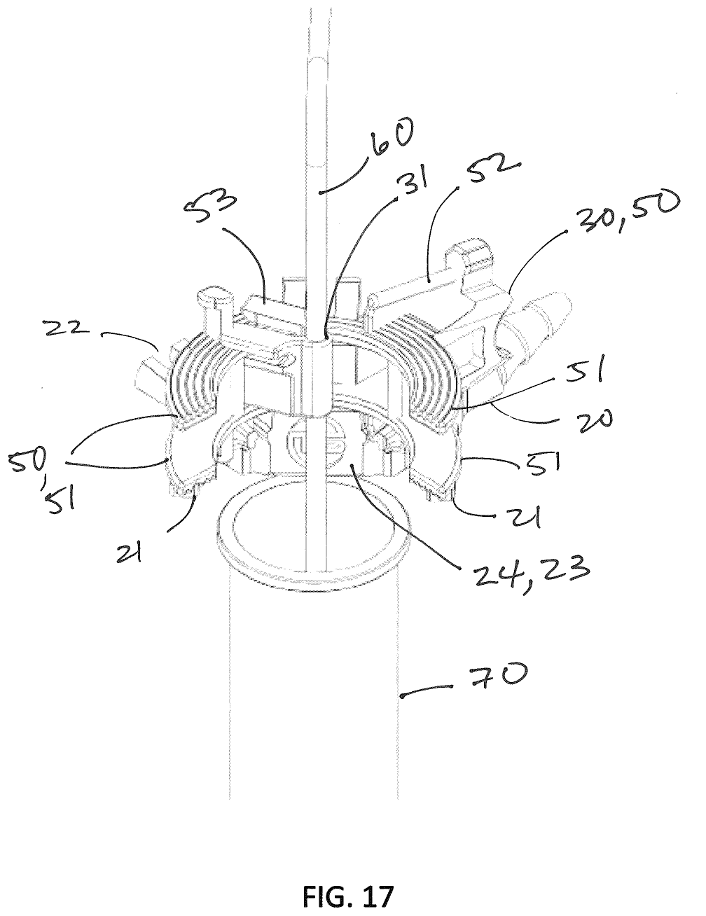

Referring now to FIG. 15 and FIG. 17, for example, in some embodiments of the surgical instrument securement system 10, the bracket 20 comprises a central plate 23 positioned between the opposing bracket arms 21, the plate including on a back side 25 a pair of opposing recesses 26 (see for example, FIG. 3 and FIG. 6) for releasable receiving the handle 71 of the surgical retractor 70.

Referring again to FIG. 15 and FIG. 17, in some embodiments of the surgical instrument securement system 10, the bracket 20 comprises an elastically deformable plate having a front side 24 and a back side 25, and first and second rails 27 extending from the back side 25 of the elastically deformable plate, each of the first and second rails 27 including at least one receptacle, the first rail including at least a first receptacle 28 and the second rail including at least a second receptacle 29, the first receptacle 28 on the first rail disposed facing the second receptacle 29 on the second rail (see for example, FIG. 3 and FIG. 6), wherein the first and second receptacles 29 are arranged to receive and secure the handle 71 of the surgical retractor 70.

In some embodiments of the surgical instrument securement system 10, the first and second opposing bracket arms 21 adjacent to and extending from the front side 24 of the elastically deformable plate are flexible such that the first and second opposing bracket arms 21 are actuatable to flex towards each other to deform the elastically deformable plate and expand a distance between the first and second rails 27 to thereby receive and secure the handle 71 of the surgical retractor 70 in the first and second receptacles 29 or remove the retractor arm from the first and second receptacles 29.

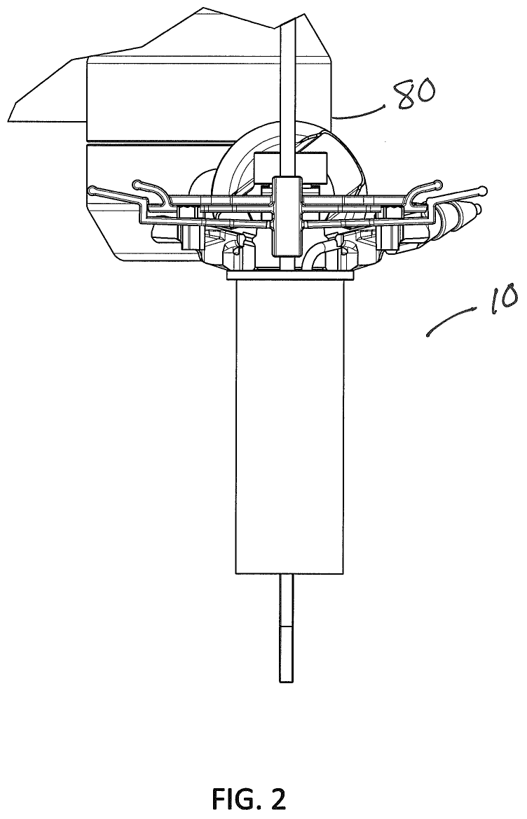

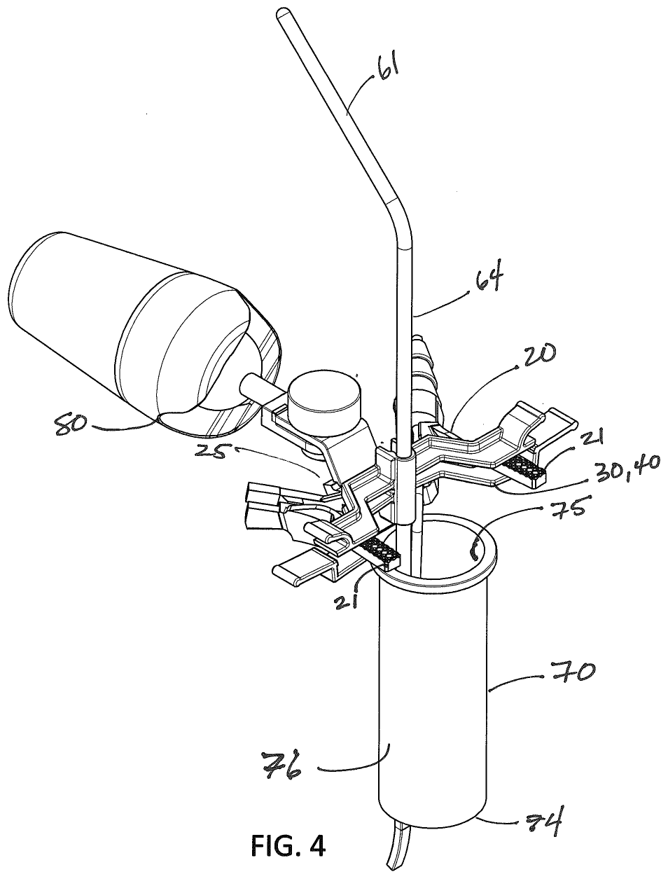

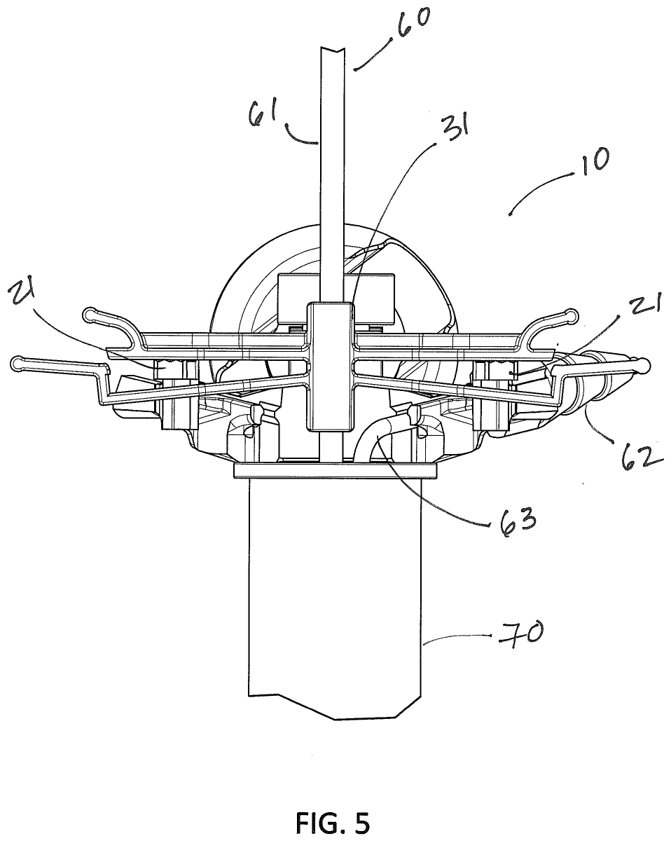

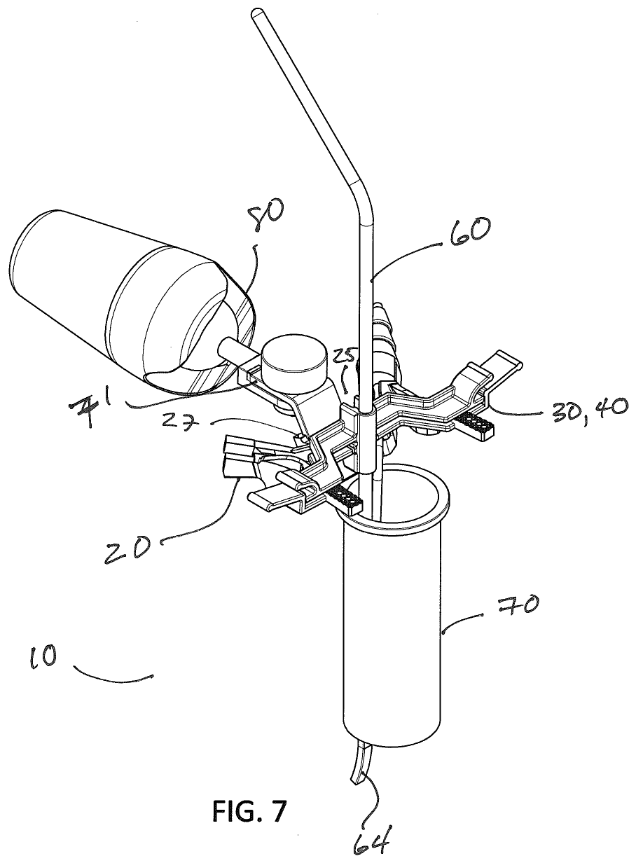

Referring now to FIG. 1-FIG. 11, in some embodiments of the surgical instrument securement system 10, the instrument securement base 30 comprises a clamp 40 comprising first and second compression wings 41, each of the first and second opposing compression wings comprising a releasable lock 42, each of the first and second compression wings 41 configured to receive and releasably compress, respectively, the first and second opposing bracket arms 21, and the instrument retention seat 31.

Referring now to FIG. 8, for example, in some embodiments of the surgical instrument securement system 10, the lock on each of the first and second compression wings 41 of the clamp 40 includes opposing and interfitting flange and tab extensions 43.

Referring again to FIG. 8, in some embodiments of the surgical instrument securement system 10, each of the first and second compression wings 41 comprises opposing compression branches comprising an upper compression branch 45 and a lower compression branch 46, and wherein the releasable lock 42 on each of the first and second compression wings 41 of the clamp 40 includes opposing and interfitting flange and tab extensions 43 positioned at a compression wing distal end 49 of each of the first and second compression wings 41.

Referring again to FIG. 8, in some embodiments of the surgical instrument securement system 10, each of the upper compression branches 45 comprises a tab extension 43 and each of the lower compression branches 46 comprises a flange extension 44, and wherein when engaged, the tab extensions 43 of each of the upper branches engage with the flange extensions 44 of each of the lower branches to compress and secure the clamp 40 to the opposing bracket arms 21.

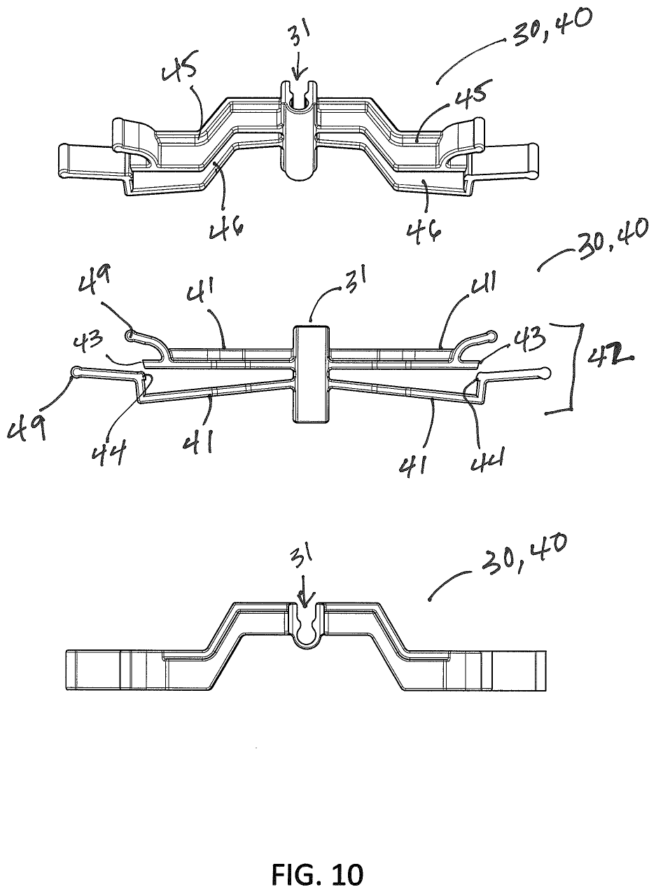

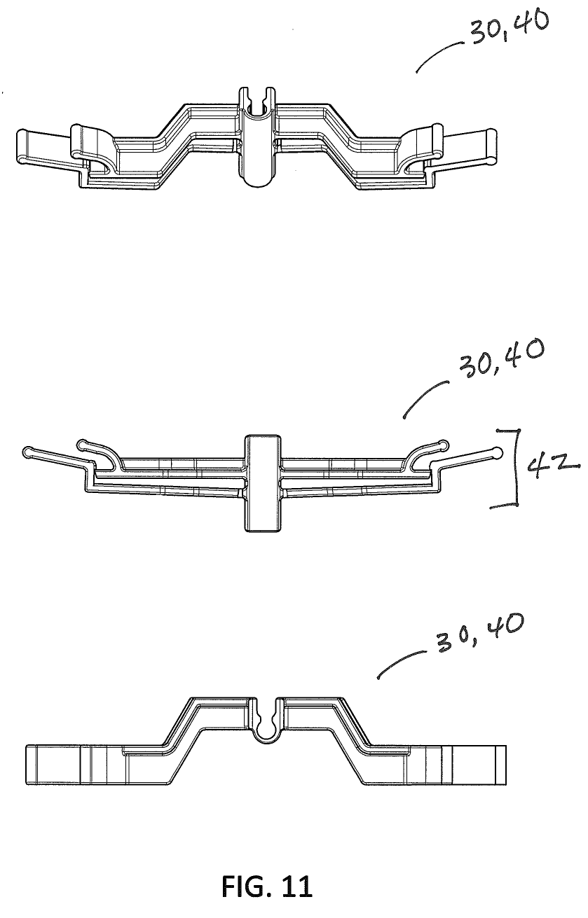

Referring now to FIG. 10 and FIG. 11, depicted are top perspective, side, and top views the clamp 40 without the bracket 20 in which the clamp 30 is in an open configuration not yet locked as shown in FIG. 10, and in which the clamp 40 is in a locked configuration as shown in FIG. 11.

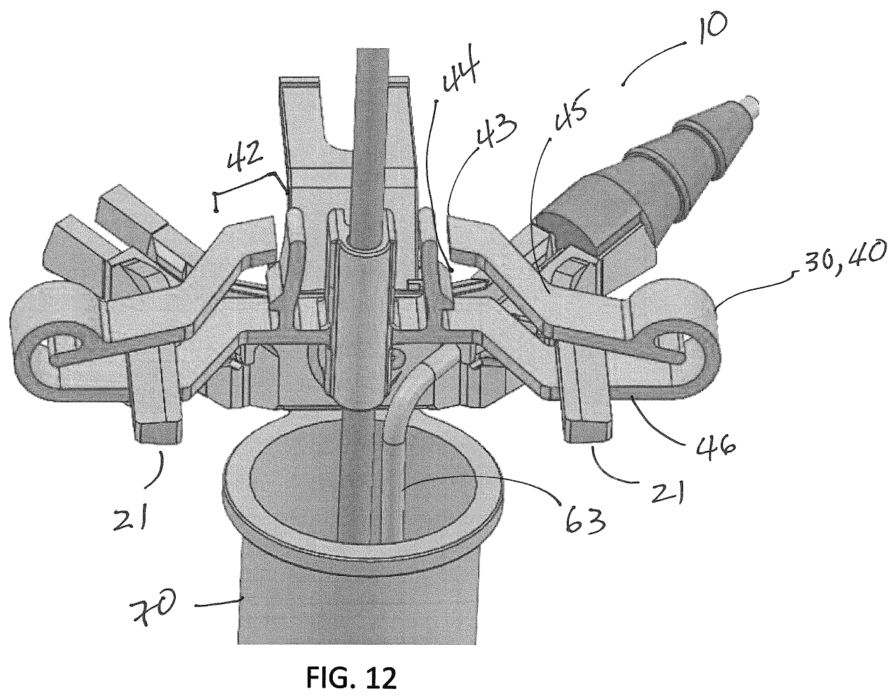

Referring now to FIG. 12, in some embodiments of the surgical instrument securement system 10, each of the first and second compression wings 41 comprises opposing compression branches comprising an upper compression branch 45 and a lower compression branch 46, and wherein the releasable lock 42 on each of the first and second compression wings 41 includes opposing and interfitting flange and tab extensions 43 positioned central to the clamp 40 adjacent the instrument retention seat 31 which is positioned between the first and second compression wings 41.

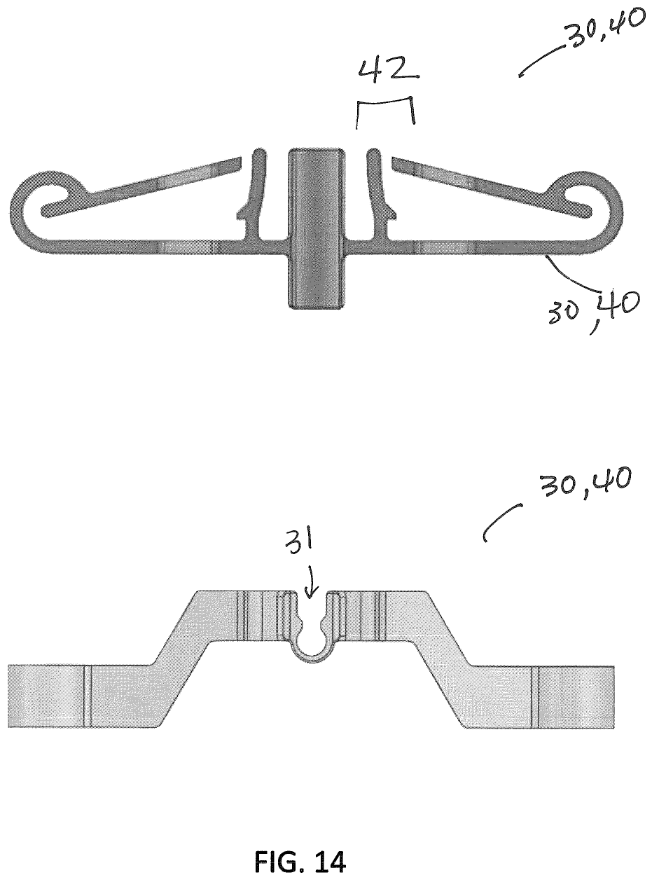

Referring now to FIG. 14, in some embodiments of the surgical instrument securement system 10, the flange of releasable lock 42 on each of the first and second compression wings 41 extends from the clamp 40 and is oriented perpendicular to the compression wings, and tab extension 43 of the releasable lock 42 on each of the first and second compression wings 41 of the clamp 40 is located at a central terminus 47 of each of the upper compression branches 45.

Referring now to FIG. 10 and FIG. 14, in the depicted alternate embodiments, the instrument retention seat 31 comprises a single recess for receiving a portion of a surgical instrument 60.

Referring again to FIG. 10 and FIG. 14, in the depicted alternate embodiments, the instrument retention seat 31 comprises at least one recess for receiving a generally cylindrical portion of a surgical instrument handle 61.

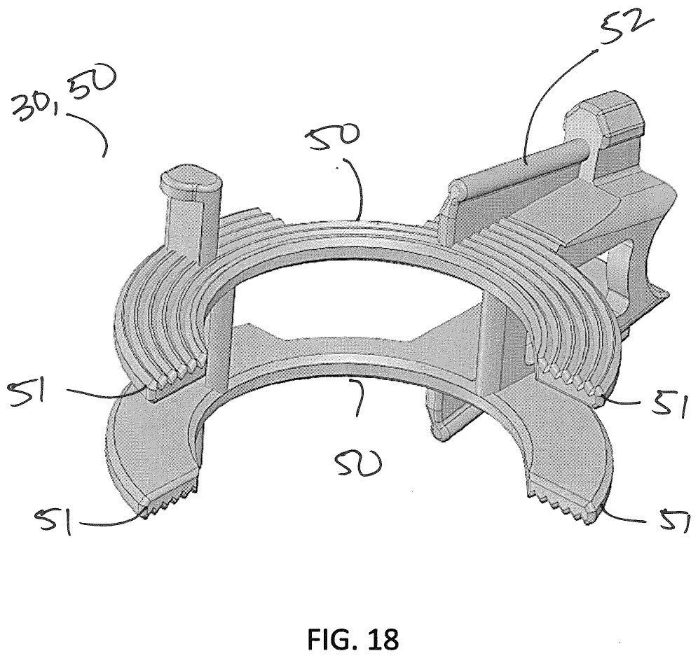

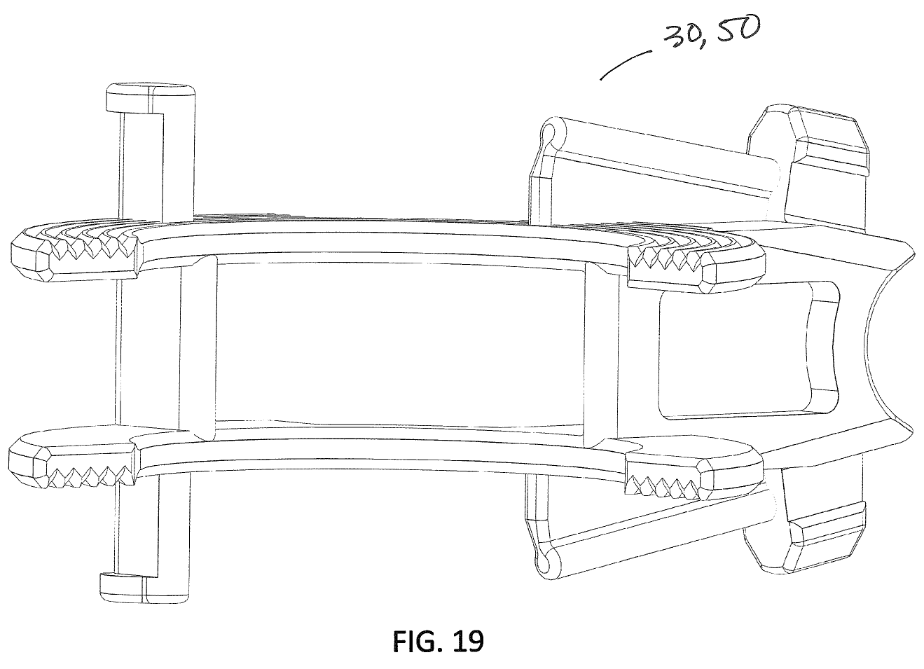

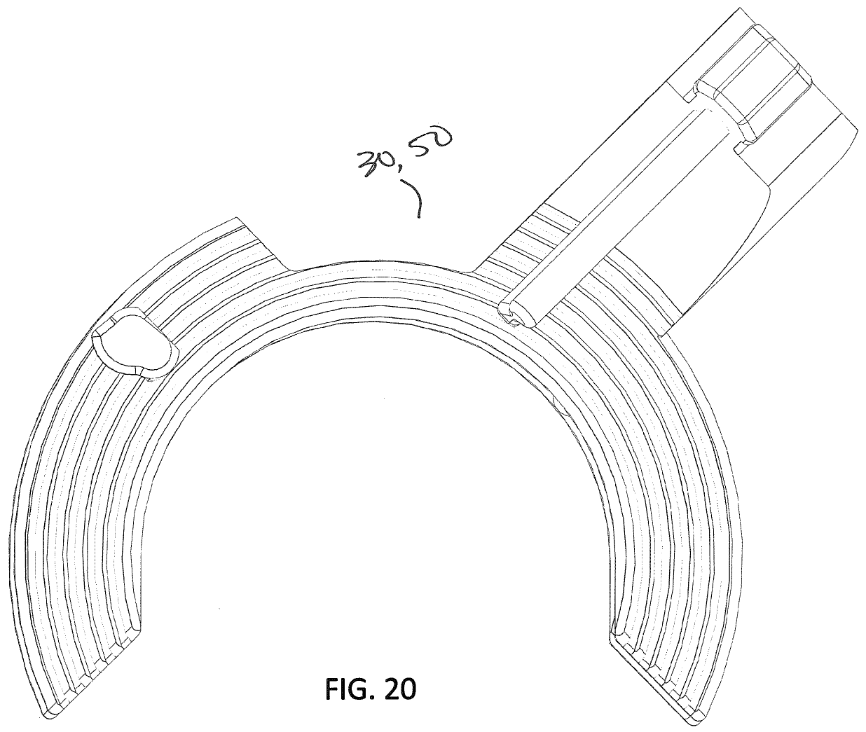

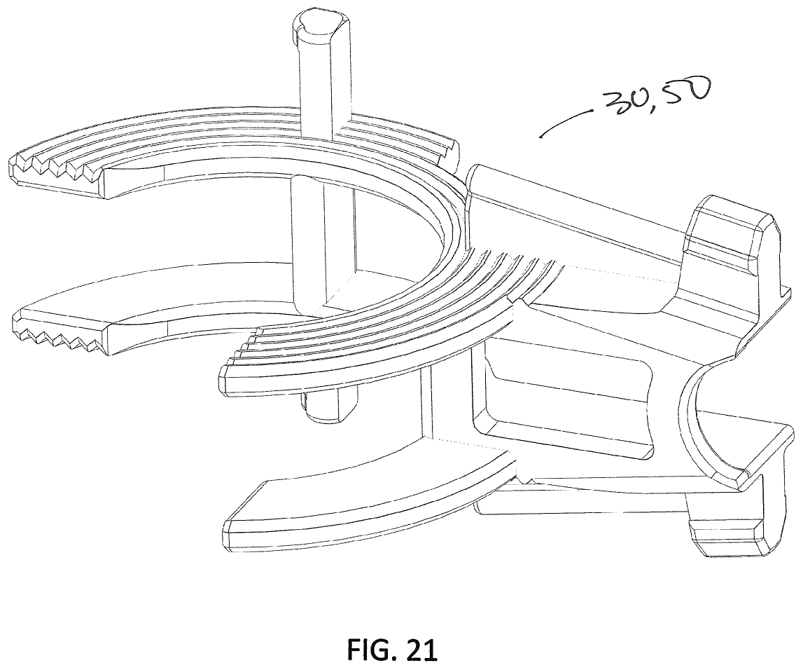

Referring now to FIG. 17-FIG. 21, in some embodiments of the surgical instrument securement system 10, the instrument securement base 30 comprises stacked arcuate supports 50 comprising upper and lower arcuate arms, each upper and lower arcuate arms 51 comprising at least one securement bar 52 for engagement with one of the emplacement 22 of the bracket arms 21; and a slidable locking collar 53 that is engageable with an arcuate arm of the stacked arcuate supports 50, the slidable locking collar 53 comprising the instrument retention seat 31 for securing at least one surgical instrument 60 to the stacked arcuate supports 50.

Referring now to FIG. 17, for example, in some embodiments of the surgical instrument securement system 10, the slidable locking collar 53 is slidably engageable along the upper arcuate arm to enable selectable positioning of an engaged surgical instrument 60 for positioning along a selected portion of the interior surface 75 of the surgical retractor 70.

In some embodiments, the disclosure provides a kit for securing a surgical instrument 60 to surgical retractor 70, comprising an embodiment of the surgical instrument securement system 10 as described herein, at least one surgical instrument 60 selected from nerve retractors, tissue shims, lighting attachments, suction attachments, or a combination thereof; and a cylindrical surgical retractor 70 comprising a handle 71.

In various embodiments at least one of the instrument securement base 30, bracket 20 and surgical instrument 60, for example but not limited to a nerve root retractor, may be formed of a polymer, or may be of metal, or a combination thereof. In various embodiments, the contact surfaces of the instrument securement base 30, including but not limited to the instrument retention seat 31, the bracket arms 21, and when used, the opposing compression branches of the compression wings, the flange and tab extensions 43 of the opposing compression branches, the stacked arcuate support arms 51, and the locking collar 53, may be textured or coated to enhance gripping and securement to prevent release of the instrument securement base 30 or the secured surgical instrument 60, such as but not limited to the depicted nerve root retractor instrument. In some embodiments, one or more of the parts may be formed of a metal or a polymer that is over molded with another polymer to confer texture or other features.

It is contemplated that the invention may be provided to surgeons or other users in the form of a kit, such kit comprising one or more surgical instrument 60 such as nerve root retractor instrument, a surgical retractor 70 such as a tubular surgical retractor 70, a bracket 20 such as one of the embodiments shown herein, and an instrument securement base 30 comprising either a clamp 40 or stacked arcuate supports 50 with a locking collar 53 such as one of the embodiments shown herein. Such kit may also include other instruments and other optional components.

In use, upon accessing the surgical field in a patient, a surgical retractor 70, surgical instrument 60, and instrument securement base 30 and bracket 20 combination are selected, and the surgical retractor 70 is inserted into position in the field according to standard surgical protocol. Thereafter, the bracket 20 is affixed to or adjacent the surgical retractor 70, depending on which bracket 20 is selected, and the instrument securement base 30 is then affixed to the bracket 20. The surgical instrument 60 is then passed into the lumen of the retractor body, and as determined by the surgeon, the distal end of the surgical instrument 60 is maneuvered for its intended use, for example the nerve root retractor is maneuvered into contact with nerve tissue below/adjacent the distal end 74 of the surgical retractor 70 so as to initially displace the tissue away from the distal lumen opening and its proximal end 73 is then secured to the instrument securement base 30 either by locking the instrument securement base 30 comprising a clamp 40 to the arms of the bracket 20 or locking using the stacked arcuate supports 50 and locking collar 53, as applicable based on the instrument securement base 30 that is chosen. As needed, the instrument may be intermittently released from the instrument securement base 30, as may be desirable to protect tissue from damage, or to remove and/or reposition the instrument or replace it with another instrument. Upon completion of the surgical procedure, one or more of the inventive components may be removed prior to removal of the surgical retractor 70, or they may be left in place and removed as a full assembly.

Embodiments of the present invention are suitable for use, in some examples, in a posterior or transforaminal approach for spinal surgery, and may be adapted for uses in other spinal surgical orientations and other surgical sites within the body.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

As used herein, the singular forms “a,” “an,” and “the” are intended to include the plural forms as well, unless the context clearly indicates otherwise. The term “proximal” as used in connection with any object refers to the portion of the object that is closest to the operator of the object or some other stated reference point, and the term “distal” refers to the portion of the object that is farthest from the operator of the object or some other stated reference point. The terms “operator” and “surgeon” each means and refers to any professional or paraprofessional who delivers clinical care to a medical patient, particularly in connection with the delivery of care.

Anatomical references as used herein are intended to have the standard meaning for such terms as understood in the medical community. Any and all terms providing spatial references to anatomical features shall have meaning that is customary in the art.

Unless otherwise indicated, all numbers expressing quantities, properties, and so forth as used in the specification, drawings and claims are to be understood as being modified in all instances by the term “about.” Accordingly, unless otherwise indicated, the numerical properties set forth in the specification and claims are approximations that may vary depending on the suitable properties desired in embodiments of the present invention. Notwithstanding that the numerical ranges and parameters setting forth the broad scope of the general inventive concepts are approximations, the numerical values set forth in the specific examples are reported as precisely as possible. Any numerical values, however, inherently contain certain errors necessarily resulting from error found in their respective measurements.

References to visualization using radiography as may be described in the exemplary techniques herein are merely representative of the options for the operator to visualize the surgical field and the patient in one of many available modalities. It will be understood by one of ordinary skill in the art that alternate devices and alternate modalities of visualization may be employed depending on the availability in the operating room, the preferences of the operator and other factors relating to exposure limits. While confirmation of instrument placement in the course of the technique is appropriate, the frequency and timing relative to the sequence of steps in the technique may be varied and the description herein is not intended to be limiting. Accordingly, more or fewer images, from more or fewer perspectives, may be collected.

One of ordinary skill will appreciate that references to positions in the body are merely representative for a particular surgical approach. Further, all references herein are made in the context of the representative images shown in the drawings. Fewer or additional instruments, including generic instruments, may be used according to the preference of the operator. Moreover, references herein to specific instruments are not intended to be limiting in terms of the options for use of other instruments where generic options are available, or according to the preference of the operator.

While the disclosed embodiments have been described and depicted in the drawings in the context of the human spine, it should be understood by one of ordinary skill that all or various aspects of the embodiments hereof may be used in in connection with other species and within any species on other parts of the body where deep access within the tissue is desirable.

While various inventive aspects, concepts and features of the general inventive concepts are described and illustrated herein in the context of various exemplary embodiments, these various aspects, concepts and features may be used in many alternative embodiments, either individually or in various combinations and sub-combinations thereof. Unless expressly excluded herein all such combinations and sub-combinations are intended to be within the scope of the general inventive concepts. Still further, while various alternative embodiments as to the various aspects, concepts and features of the inventions (such as alternative materials, structures, configurations, methods, devices and components, alternatives as to form, fit and function, and so on) may be described herein, such descriptions are not intended to be a complete or exhaustive list of available alternative embodiments, whether presently known or later developed.

Those skilled in the art may readily adopt one or more of the inventive aspects, concepts and features into additional embodiments and uses within the scope of the general inventive concepts, even if such embodiments are not expressly disclosed herein. Additionally, even though some features, concepts and aspects of the inventions may be described herein as being a preferred arrangement or method, such description is not intended to suggest that such feature is required or necessary unless expressly so stated. Still further, exemplary or representative values and ranges may be included to assist in understanding the present disclosure; however, such values and ranges are not to be construed in a limiting sense and are intended to be critical values or ranges only if so expressly stated.

I claim:

1. A surgical instrument securement system for use with a surgical retractor, comprising:
(a) a bracket comprising first and second opposing bracket arms; and
(b) an instrument securement base that is releasably engageable with one or both of the first and second opposing bracket arms, the instrument securement base comprising an instrument retention seat, the instrument retention seat configured to retain a portion of at least one surgical instrument,
wherein, the bracket and instrument securement base, when assembled, are configured to secure the at least one surgical instrument to a surgical retractor that has an elongate body having a proximal end, a distal end, an interior surface, and an exterior surface, and a retractor axis defined along the elongate body from the proximal to the distal end, and
wherein when the at least one surgical instrument is releasably secured to the surgical retractor, the at least one surgical instrument is positioned adjacent at least a portion of the interior surface of the surgical retractor.

2. The surgical instrument securement system according to claim 1, wherein the surgical retractor is generally cylindrical in shape.

3. The surgical instrument securement system according to claim 1, wherein the surgical retractor includes affixed clip extensions for contacting and displacing soft tissue away from the surgical field.

4. The surgical instrument securement system according to claim 1, wherein at least one of the first and second opposing bracket arms includes an emplacement for retaining one or more of surgical instruments selected from nerve retractors, tissue shims, lighting attachments, suction attachments, or a combination thereof.

5. The surgical instrument securement system according to claim 1, wherein the bracket is integral with the surgical retractor.

6. The surgical instrument securement system according to claim 1, wherein the bracket is configured to releasably attach to a handle of the surgical retractor.

7. The surgical instrument securement system according to claim 6, wherein the bracket comprises a central plate positioned between the opposing bracket arms, the plate including on a back side a pair of opposing recesses for releasable receiving the handle of the surgical retractor.

8. The surgical instrument securement system according to claim 6, wherein the bracket comprises an elastically deformable plate having a front side and a back side, and first and second rails extending from the back side of the elastically deformable plate, each of the first and second rails including at least one receptacle, the first rail including at least a first receptacle and the second rail including at least a second receptacle, the first receptacle on the first rail disposed facing the second receptacle on the second rail, wherein the first and second receptacles are arranged to receive and secure the handle of the surgical retractor.

9. The surgical instrument securement system according to claim 6, wherein the first and second opposing bracket arms adjacent to and extending from the front side of the elastically deformable plate are flexible such that the first and second opposing bracket arms are actuatable to flex towards each other to deform the elastically deformable plate and expand a distance between the first and second rails to thereby receive and secure the handle of the surgical retractor in the first and second receptacles or remove the retractor arm from the first and second receptacles.

10. The surgical instrument securement system according to claim 1, wherein
the instrument securement base comprises a clamp comprising first and second compression wings, each of the first and second opposing compression wings comprising a releasable lock, each of the first and second compression wings configured to receive and releasably compress, respectively, the first and second opposing bracket arms, and the instrument retention seat.

11. The surgical instrument securement system according to claim 10, wherein the releasable lock on each of the first and second compression wings of the clamp includes opposing and interfitting flange and tab extensions.

12. The surgical instrument securement system according to claim 11, wherein each of the first and second compression wings comprises opposing compression branches comprising an upper compression branch and a lower compression branch, and wherein the releasable lock on each of the first and second compression wings of the clamp includes opposing and interfitting flange and tab extensions positioned at a distal end of each of the first and second compression wings.

13. The surgical instrument securement system according to claim 12, wherein each of the upper compression branches comprises a tab extension and each of the lower compression branches comprises a flange extension, and wherein when engaged, the tab extensions of each of the upper branches engage with the flange extensions of each of the lower branches to compress and secure the clamp to the opposing bracket arms.

14. The surgical instrument securement system according to claim 10, wherein each of the first and second compression wings comprises opposing compression branches comprising an upper compression branch and a lower compression branch, and wherein the releasable lock on each of the first and second compression wings includes opposing and interfitting flange and tab extensions positioned central to the clamp adjacent the instrument retention seat which is positioned between the first and second compression wings.

15. The surgical instrument securement system according to claim 14, wherein the flange of releasable lock on each of the first and second compression wings extends from the clamp and is oriented perpendicular to the compression wings, and tab extension of the releasable lock on each of the first and second compression wings of the clamp is located at a central terminus of each of the upper compression branches.

16. The surgical instrument securement system according to claim 10, wherein the instrument retention seat comprises a single recess for receiving a portion of a surgical instrument.

17. The surgical instrument securement system according to claim 10, wherein the instrument retention seat comprises at least one recess for receiving a generally cylindrical portion of a surgical instrument handle.

18. The surgical instrument securement system according to claim 1, wherein
the instrument securement base comprises:
(a) stacked arcuate supports comprising upper and lower arcuate arms, each upper and lower arcuate arms comprising at least one securement bar for engagement with one of the emplacement of the bracket arms; and
(b) a slidable locking collar that is engageable with an arcuate arm of the stacked arcuate supports, the slidable locking collar comprising the instrument retention seat for securing at least one surgical instrument to the stacked arcuate supports,
wherein, the slidable locking collar is slidably engageable along the upper arcuate arm to enable selectable positioning of an engaged surgical instrument for positioning along a selected portion of the interior surface of the surgical retractor

19. A kit for securing a surgical instrument to surgical retractor, comprising:
(a) a surgical instrument securement system according to claim 1;
(b) at least one surgical instrument selected from nerve retractors, tissue shims, lighting attachments, suction attachments, or a combination thereof; and
(c) a cylindrical surgical retractor comprising a handle.

20. A method for accessing the surgical field in a patient, comprising:
(a) providing surgical instrument securement system according to claim 1;
(b) affixing the surgical instrument securement system to a tubular surgical retractor in a surgical field to provide a surgical retractor assembly;
(c) passing a selected surgical instrument into the tubular surgical retractor, and as determined by the surgeon, the maneuvering a distal end of the surgical instrument for into contact with tissue below/adjacent a distal end of the surgical retractor so as to initially displace soft tissue away from the distal end of the surgical retractor;
(d) securing the instrument securement by engagement with the instrument retention seat of the instrument securement base;
(e) locking the instrument to the instrument retention seat;
(f) optionally, intermittently releasing the instrument from the instrument securement base; and
(g) upon completion of the surgical procedure, removing the surgical retractor assembly.
